(19) United States
(12) Patent Application Publication
(10) Pub. No.: US 2005/0234465 A1
(43) Pub. Date: Oct. 20, 2005

(54) Guided saw with pins

(76) Inventors: Daniel L. McCombs, Memphis, TN (US); Lauralan Terrill-Grisoni, Cordova, TN (US)

Correspondence Address: CHIEF PATENT COUNSEL;SMITH & NEPHEW, INC., 1450 BROOKS ROAD, MEMPHIS, TN, 38116, US

(21) Appl. No.: 11/095,292

(22) Filed: Mar. 31, 2005

Related U.S. Application Data
(60) Provisional application No. 60/558,207, filed on Mar. 31, 2004.

(51) Int. Cl. A61B 17/58

(52) U.S. Cl. 606/88

(57) ABSTRACT

Embodiments of improved systems and processes for cutting bones. Embodiments of improved systems and processes for resecting a femoral bone are provided, the system comprising a guiding device adapted to be connected to the femoral bone, a cutting device having a cutting element adapted to resect bone, and a link adapted to connect to the guiding device to the cutting device, wherein the link is adapted to constrain movement of the cutting device. Embodiments of improved systems and processes for cutting knee bones during computer assisted knee surgery are provided, comprising at least one fiducial associated with the guiding device and a tracking functionality capable of tracking a position and orientation of the at least one fiducial.

### CROSS-REFERENCE TO RELATED APPLICATIONS

This application claims the benefit of U.S. Provisional Application Ser. No. 60/558,207 entitled “Guided Saw with Pins” filed on Mar. 31, 2004, the entire content of which is incorporated herein.

### TECHNICAL FIELD

Systems, devices and methods for preparing bones for installing joint implants during joint replacement surgery. Systems and methods for cutting bones for installing joint implants during joint replacement surgery. Particularly, systems and methods for resecting femoral or tibial bones, or both, during knee replacement surgery, such as total knee arthroplasty. Systems comprising guided cutting surgical cutting instruments, such as surgical saws, for use during computer assisted surgical (CAS) procedures, such as computer assisted joint replacement surgery. Methods of guiding instruments for cutting bones, such as saws, during CAS procedures.

### BACKGROUND

Joint implants, also referred to as joint prostheses, joint prosthetic implants, joint replacements, or prosthetic joints, are long-term surgically implantable devices that are used to partially or totally replace diseased or damaged joints within the musculoskeletal system of a human or an animal. Damaged joints repaired during such procedures include, but are not limited to, a knee, a hip, a shoulder, an ankle, or an elbow joint. Since their first introduction into clinical practice in the 1960s, joint implants have improved the quality of life of many patients.

Knee arthroplasty is a procedure for replacing components of a knee joint damaged by trauma or disease. During this procedure, a surgeon removes a portion of one or more bones forming the knee joint and installs prosthetic components to form the new joint surfaces. In the United States, surgeons perform annually approximately 250,000 total knee arthroplasties (TKAs), or total replacements of a knee joint. Thus, it is highly desirable to improve this popular technique to ensure better restoration of knee joint function and to shorten the patients' recovery time.

### Structure of the Human Knee Joint

The structure of the human knee joint is described in many standard manuals. One example is “Questions and Answers About Knee Problems” (2001), National Institute of Arthritis and Musculoskeletal and Skin Diseases (NIAMS) Information Clearinghouse National Institutes of Health (NIH), Bethesda, Md.). The human knee joint includes essentially four bones. The lower extremity of the femur, or distal femur, attaches by ligaments and a capsule to the proximal tibia. The distal femur contains two rounded oblong eminences, the condyles, separated by an intercondylar notch. The tibia and the femur do not interlock, but meet at their ends. The femoral condyles rest on the condyles of the proximal tibia. The fibula, the smaller shin bone, attaches just below the tibia and is parallel to it. The patella, or the knee cap, is at the front of the knee, protecting the joint and providing extra leverage. A patellar surface is a smooth shallow articular depression between the femoral condyles at the front. Cartilage lines the surfaces of the knee bones, cushions them, and minimizes friction. Two C-shaped menisci, or meniscal cartilage, lie between the femur and the tibia, serve as pockets for the condyles, and stabilize the knee. Knee ligaments connect the knee bones, cover and stabilize the joint. The knee ligaments include the patellar ligament, the medial and lateral collateral ligaments, and the anterior (ACL) and posterior (PCL) cruciate ligaments. The medial collateral ligament (MCL) provides stability to the inner (medial) part of the knee. The lateral collateral ligament (LCL) provides stability to the outer (lateral) part of the knee. The anterior cruciate ligament (ACL), in the center of the knee, limits rotation and the forward movement of the tibia. The posterior cruciate ligament (PCL), also in the center of the knee, limits backward movement of the tibia. Ligaments and cartilage provide the strength needed to support the weight of the upper body and to absorb the impact of exercise and activity. Tendons, muscle, and cartilage are also important for joint stabilization and functioning. Some examples of the tendons are popliteus tendon, which attaches popliteus muscle to the bone. Pes anserinus is the insertion of the conjoined tendons into the proximal tibia, and comprises the tendons of the sartorius, gracilis, and semitendinosus muscles. The conjoined tendon lies superficial to the tibial insertion of the MCL. The iliotibial band extends from the thigh down over the knee and attaches to the tibia. In knee flexion and extension, the iliotibial band slides over the lateral femoral epicondyle. The knee capsule surrounds the knee joint and contains lubricating fluid synovium.

A healthy knee allows the leg to move freely within its range of motion while supporting the upper body and absorbing the impact of its weight during motion. The knee has generally six degrees of motion during dynamic activities: three rotations (flexion/extension angulations, axial rotation along the long axis of a large tubular bone, also referred to as interior/exterior rotation, and varus/valgus angulations); and three translations (anterior/posterior, medial/lateral, and superior/inferior).

### Total Knee Arthroplasty

A total knee arthroplasty, or TKA, replaces both the distal femur and the proximal tibia of the damaged or diseased knee with artificial components made of various materials, including, but not limited to, metals, ceramics, plastics, or their combinations. These prosthetic knee components are attached to the bones, and the existing soft tissues are used to stabilize the artificial knee. During TKA, after preparing and anesthetizing the patient, the surgeon makes a long incision along the front of the knee and positions the patella to expose the joint. After exposing the ends of the bones, the surgeon removes the damaged tissue and cuts, or resects, the portions of the tibial and femoral bones to prepare the surfaces for installation of the prosthetic components.

After bone preparation, the knee is tested with the trial components. Soft-tissue balancing, including any necessary surgical release or contraction of the knee ligaments, also referred to as ligament balancing, and other soft tissues, is performed to ensure proper post-operative functioning of the knee. After ligament balancing and proper selection of the components, the surgeon installs and secures the tibial and femoral components. The patella is typically resurfaced after installation of the tibial and femoral component, and a small plastic piece is often placed on the rear side, where it will cover the new joint. After installation of the knee prosthesis, the knee is closed according to conventional surgical procedures. Post-operative rehabilitation starts shortly after the surgery to restore the knee's function.

In order to ensure good post-operative functioning of the prosthetic knee, accurate positioning, and alignment of the prosthetic knee components is necessary. Improper positioning and misalignment of the prosthetic knee components commonly cause prosthetic knees to fail. This leads to revision surgeries and increases the risks associated with knee replacement. Many patients requiring prosthetic knee components are elderly and highly prone to the medical complications resulting from multiple surgeries. Thus, revision surgeries greatly increase the medical costs associated with the restoration of the knee function.

In order to prevent premature, excessive, or uneven wear of the artificial knee, the surgeon must implant the prosthetic device so that its multiple components articulate at exact angles, and are properly supported and stabilized by accurately balanced knee ligaments. Therefore, correctly preparing the bone for installation of the prosthetic components by precisely determining and accurately performing all the required bone cuts is vital to the success of TKA.

To properly prepare femoral surfaces to accept the femoral and tibial components of the prosthetic knee, the surgeon needs to accurately determine the position of and perform multiple cuts. Femoral cuts during TKA, include, but are not limited to, a transversely directed distal femoral cut, an axially directed anterior femoral cut, an axially directed posterior femoral cut, anterior and posterior chamfer femoral cuts, a trochlear recess cut, or any combination or variation of those. Preparation of the tibia for installation of the tibial component may also involve multiple cuts. The surgeons generally rely heavily on their experience to determine where the bone should be cut. The surgeon may use various measuring and indexing devices to determine the location of the cut, and various devices, such as, but not limited to, cutting guides, jigs, blocks or templates, to direct the saw blades in the bone cuts. After determining the desired position of the cut, the surgeon usually stabilizes the cutting guide, jig, block or template at the patient by attaching the device to the bone using appropriate fastening mechanisms, including, but not limited to, pins and screws. For stabilization, the device can also be associated with the structures already affixed to the bone, such as intramedullary rods, femoral pins, and the like. Typically, after stabilizing the device at the bone, the surgeon uses it to direct the saw blade in the plane of the cut. A TKA procedure may involve sequentially attaching to the bone and properly positioning a series of cutting guides, guides, jigs, blocks or templates, each adapted for a specific task.

### Minimally Invasive Surgery

One recent development in joint replacement is the so-called “minimally invasive surgery” (MIS) techniques, or, generally, the surgical techniques that minimize the size of the surgical incision and trauma to tissues. MIS is generally less intrusive than conventional surgery, thereby shortening both surgical time and recovery time. To achieve the goals of MIS, it is necessary to modify the traditional implants and instruments, including bone preparation systems and instruments, so that they do not require long surgical cuts and extensive exposure of the internal knee structures. The conventional bone preparation systems and instruments are typically not suitable for minimally invasive surgery. The conventional bone preparation instruments are generally too large to be placed in a small incision, too cumbersome to use, and require additional mechanical referencing devices for proper positioning and adjustment, and guiding devices for accurate operation. Preferably, the systems and devices adapted for MIS can be installed and adjusted with minimal trauma to the knee's tissues and allow the surgeon to perform the cuts quickly and efficiently without compromising the accuracy of the resection. In one aspect, to adapt the traditional bone preparation surgical systems and techniques to MIS, it is desirable to decrease their size and the number of components. In another aspect, it is desirable to minimize the number of the surgical steps required to accurately cut the bones in preparation for installation of the prosthetic knees.

### Computer Assisted Surgery

Another recent development in joint replacement is computer assisted surgery (CAS). The CAS systems and processes use various imaging and tracking devices and combine the image information with computer algorithms to track the position of the patient's anatomy, surgical instruments, prosthetic components, virtual surgical constructs such as body and limb axes, and other surgical structures and components. The CAS systems and processes can provide useful data throughout the surgery on predicted or actual position and orientation of body parts, surgically related items, implants, and virtual constructs for use in navigation, assessment, and otherwise performing surgery or other operations. The CAS systems and processes use this data to make highly individualized recommendations on a number of parameters, including, but not limited to, patient's positioning, the most optimal surgical cuts, adjustment of soft tissues, and prosthetic component selection and positioning.

Several manufacturers currently produce CAS navigation systems for orthopedic procedures, such as joint replacement. The TREON™ and ION™ systems with FLUORONAV™ software manufactured by Medtronic Surgical Navigation Technologies, Inc. are examples of such systems. The BrainLAB VECTORVISION™ system is another example of such a surgical navigation system. Systems and processes for accomplishing computer-aided surgery are also disclosed in U.S. Ser. No. 10/084,012, filed Feb. 27, 2002 and entitled “Total Knee Arthroplasty Systems and Processes”; U.S. Ser. No. 10/084,278, filed Feb. 27, 2002 and entitled “Surgical Navigation Systems and Processes for Unicompartmental Knee Arthroplasty”; U.S. Ser. No. 10/084,291, filed Feb. 27, 2002 and entitled “Surgical Navigation Systems and Processes for High Tibial Osteotomy”; International Application No. US02/05955, filed Feb. 27, 2002 and entitled “Total Knee Arthroplasty Systems and Processes”; International Application No. US02/05956, filed Feb. 27, 2002 and entitled “Surgical Navigation Systems and Processes for Unicompartmental Knee Arthroplasty”; International Application No. US02/05783 entitled “Surgical Navigation Systems and Processes for High Tibial Osteotomy”; U.S. Ser. No. 10/364,859, filed Feb. 11, 2003 and entitled “Image Guided Fracture Reduction,” which claims priority to U.S. Ser. No. 60/355,886, filed Feb. 11, 2002 and entitled “Image Guided Fracture Reduction”; U.S. Ser. No. 60/271,818, filed Feb. 27, 2001 and entitled “Image Guided System for Arthroplasty”; and U.S. Ser. No. 10/229,372, filed Aug. 27, 2002 and entitled “Image Computer Assisted Knee Arthroplasty”, the entire contents of each of which are incorporated herein by reference as are all documents incorporated by reference therein.

Many orthopedic surgeries, including, but not limited to TKR and other types of knee arthroplasties, involve the use of a wide array of instrumentation, systems and other surgical items. Surgical instrumentation, systems and items include, but are not limited to: sleeves to serve as entry tools, working channels, drill guides and tissue protectors; scalpels; entry awls; guide pins; reamers; reducers; distractors; guide rods; endoscopes; arthroscopes; saws and other bone cutting implements; drills; screwdrivers; awls; taps; osteotomes and wrenches. In many surgical procedures, including orthopedic surgeries, such as joint replacement surgeries, it may be desirable to associate some or all of these items with an item, for example, but not limited to, a guide or a handle, that incorporates a surgical reference, allowing the instrument to be used with a CAS navigation system. In particular, systems, instruments, devices and methods for performing accurate bone cuts are desirable that can be computer navigated and positioned after taking into account the feedback from the computer functionality.

### Conventional Bone Preparation Systems

Conventional bone preparation systems, instruments and devices, such as guides, blocks, jigs, or templates, suffer from drawbacks that make it difficult to adapt them for MIS, CAS, or both. For example, the known systems often involve numerous components and parts. This complicates the operation, and, particularly, makes it difficult to adapt the system to CAS, MIS, or both. It demands a great deal of resources for the CAS system and its user to register, track, and navigate the numerous components and parts. MIS applications also favor simpler surgical systems and instruments with fewer components, because they can be more readily reduced in size than the conventional systems, and/or adapted for operation through minimally invasive incisions.

Conventional bone preparation systems and methods typically include instruments and devices, such as, but not limited to, guides, jigs, blocks and templates, for directing or controlling the cutting implement, or blade. Perfuming accurate cuts using these guiding instruments and devises demands significant skill from the surgeon. If he or she loses control, the instrument can change position or slip, jeopardizing the accuracy of the resection and the safety of the patient. Another problem is the debris generated as a wear product between the guiding device and the blade. Such debris are typically metal, because the guiding devices, blades, or both, are commonly manufactured from metal. The debris contaminate the surgical field and can lead to patient trauma, infection, or both.

One example of known guiding systems suffering from the above drawbacks is a known cutting guide assembly for tibial osteotomy, specifically, for removing a wedge-shaped segment of the tibia for correcting varus (bowlegged) or valgus (knock-kneed) deformities of the tibia. The assembly comprises two pairs of guide pins inserted into a patient's tibia at a predetermined angle, thereby defining two intersecting guide planes. A saw blade is guided along the surfaces of each pair of guide pins to cut a wedge-shaped segment from the tibia along the intersecting planes. The pins are accurately inserted into the bone through a guide block with appropriate bores. The saw can be additionally guided by a guide plate attached to the pins. Thus, in this known device, surgeon glides the saw blade against the guiding pins or other guiding structures, thereby generating debris.

Another example is a system that uses multiple components for guiding a saw when preparing a knee joint for a prosthesis during TKR. The system includes a single guide member for use in resecting the distal femoral condyles, the proximal tibia, and the distal femur. The guide member is used with the knee joint in flexion and has three pairs of parallel guide slots which for triplanar bone resection. The guide member also has an alignment opening for a guide rod, which has a 900 bend and is inserted into the femur. Thus, the system employs multiple components, such as guiding blocks, slots or other structures against which to cut a saw, which leads to debris generation, and also employs a separate guiding rod for attachment to the bone. The system is poorly adaptable for MIS or CAS.

To simplify bone resection, some conventional systems attach the whole saw directly to the bone. One such system is a continuously driven flexible chain saw for cutting a disk or bone out of the skull, or trepanning. The device comprises a guard/stop structure adapted for cutting sections out of the skull bones. The saw is trained around a guard member, and a ball shaped contact head bears on the outer surface of the skull and holds the guard upwardly against the inner surface. For trepanation, an aperture in the skull is drilled, the guide is inserted through the and positioned beneath the inner surface of the skull, and the saw is moved about in any desired manner to cut a section from the skull, with the guard member tracking on the inner surface of the skull, and the ball-shaped stop member holding the guard closely against the under surface. This known system is not adapted for making planar cuts in large tubular bones, and is not adapted for cutting the bones during orthopedic procedures. In this known system, the saw is not constrained by and guided in a predetermined cutting plane. Rather, in this known system, the saw moves freely along the skull surface guided by the surgeon. In contrast, in the orthopedic procedures, it is necessary to perform accurate planar cuts along pre-determined resection planes. Thus, the system for trepanning is not adapted for orthopedic surgeries.

A device for cutting bones during orthopedic procedures that mounts directly on the patient's femur with pins is also known. The devise comprises, among other things, a mounting rail parallel to the femur, a support mounted on the rail and moving perpendicularly to the rail, a saw carriage linearly sliding in a housing, and a saw with a blade extending in the direction of a linear movement. The saw and the saw carriage are manually moved by the surgeon. The carriage can be secured on the devise in several positions for making various femoral cuts. Although this device does not employ additional structures for guiding the saw blade, the device is complex and comprises multiple parts, such as a separate mounting rail for attaching the instrument to the bones. The device is not adapted for use with minimally invasive or computer assisted surgical procedures.

In view of the foregoing drawbacks of the conventional systems, and poor prospects for adapting them for modern orthopedic techniques, improved systems are needed for performing accurate bone cuts during joint replacement surgeries, such as, but not limited to, TKA. Such desired improved systems would preferably be particularly well adapted for use in MIS, CAS, or both. They would preferably be less complex than the conventional systems and devices, and preferably allow the surgeon to minimize the size of the surgical incision and tissue damage, thereby reducing the surgical repairs and shortening the recovery time. Improved systems for performing accurate bone cuts are also needed that preferably minimize damage to the bone and soft tissues during installation and operation, and that can preferably be positioned and installed at the bone without the encumbrances of mechanical referencing devices. Improved systems for performing accurate bone cuts would be preferred that allow the surgeon to accurately cut the bone without using the additional devices for directing the cutting blades, and generate less metal or other material debris during operation than the conventional systems.

Further, improved systems would preferably comprise cutting instruments whose position can be precisely controlled before and after installation, so that it is possible to place them accurately in the desired location suggested by the navigation system. Improved systems for performing accurate bone cuts would preferably be adapted for incorporating a surgical reference, allowing the instrument to be used with a CAS navigation system. Preferably, the improved systems would be useful for performing one or more accurate femoral bone cuts during knee replacement procedures, but would not be limited to this application.

In general, improved systems for performing accurate bone cuts are needed, particularly for use in TKA and other knee arthroplasties, that feature at least some of the following properties: they are easy to use and manufacture, minimize tissue damage, simplify surgical procedures, are robust, can withstand multiple surgeries and required sterilization treatments, are versatile, allow for faster healing with fewer complications, require less post-surgical immobilization, are simple to use so as to require less operator training, and also less costly to produce and operate.

### SUMMARY

Embodiments of the present invention provide improved systems and methods for accurately cutting bones in orthopedic surgical procedures, such as TKA. In one aspect, the improved systems comprise a cutting device having a cutting element adapted to resect, or cut, bone, a guiding device adapted to be connected to the bone, and a link adapted to connect to guiding device to the cutting device, wherein the link is adapted to constrain movement of the cutting device; whereby the cutting device is permitted to be moved in a manner that causes the cutting element to resect the bone.

In some embodiments of the improved systems, the cutting device is a saw adapted for positioning at a patient's bone with the help of one or more guiding devices adapted for association with the patient's bone and for engaging the saw. The saw, in turn, is adapted to be engaged by the one or more guiding devices. The one or more guiding devices engage the guided saw and control its position. The one or more guiding devices can either control the saw in a fixed position or be adapted to control the movement of the saw along a desired path, such as the the bone cut.

In one exemplary embodiment, the guiding device is one or more pins adapted for insertion into and fixation in the patient's bone and for engaging the saw. When engaged by the one or more pins, the saw is attached in the fixed position at the bone. The saw blade is moveable relative to the saw and the patient's bone. In a preferred embodiment, the blade is adapted to pivot in the plane of the cut. For cutting the bone, a user, such as a surgeon, guides the saw and positions it at the bone with the pins, and manipulates the saw to move the blade to perform the cut.

In another exemplary embodiment, the guiding device is a guiding block that is adapted for attachment to the patient's bone and for engaging the saw and guiding it along a desired path. The guiding block comprises at least one guiding feature corresponding generally to a resection to be formed on the bone. The block and the cutting device are so adapted as to when the cutting device, such as, but not limited to, a saw, is engaged by and guided by the block, the cutting element is positioned in guided in the desired resection in the patient's bone.

Thus, in one aspect, embodiments of the improved systems of the present invention allow the surgeons to accurately cut bone during orthopedic procedures, such as TKR, without using cutting blocks, guides or other similar devices, for directing the cutting blades. This considerably reduces the complexity of the system, and makes the system easier to manufacture and operate. In another aspect, embodiments of the improved systems of the present invention allow the user, such as surgeon, improved control of the cutting device as compared to the conventional systems that allow the surgeon to only direct the cutting element, rather than the entire device, during the cut. This reduces the surgical mistakes and the patient's trauma. In one more aspect, embodiments of the systems of the present invention allow the user to perform accurate bone cuts without the need for multiple cutting blocks. In one more aspect, embodiments of the improved systems provided herein advantageously generate less metal or other material debris during operation as compared to the conventional systems. This reduces infection and trauma to the patient.

Methods of cutting a bone during surgery using systems and devices according to aspects and embodiments of the present invention generally comprise the following steps, not necessarily in the listed order: stabilizing a guiding device with respect to a patient; engaging the cutting device with the guiding device; and manipulating the cutting device to cut, or resect, the bone. Methods according to certain aspects and embodiments of the present invention can further comprise the step of disengaging the cutting device from the guiding device. They can further comprise the step of removing the guiding device from the patient.

An advantage of certain embodiments of the improved systems provided herein over conventional systems is that they are preferably particularly well adapted for use in MIS, CAS, or both. Certain aspects and embodiments of the improved systems provided herein allow the surgeon to minimize the size of the surgical incision and,tissue damage, thereby reducing the surgical repairs and shortening the recovery time, and minimize damage the bone and soft tissues during installation and operation. In one embodiment, the improved systems for accurately cutting bones are adapted for CAS. In this embodiment, the one or more guiding devices, or the cutting device, or both incorporate one or more surgical references for CAS navigation.

Some aspects and embodiments of the improved systems provided herein are particularly well-suited for incorporating a surgical reference, such as one or more fiducials, thus allowing the instrument to be used with a CAS navigation system. At the same time, other aspects and embodiments of the improved systems provided herein allow the instrument to be used with a CAS navigation system without overextending its resources, and simplify registering, tracking, and navigating by the CAS navigation system. Certain embodiments of the improved systems provided herein can be positioned and installed at the bone without the encumbrances of mechanical referencing devices. The position of the improved systems for performing accurate bone cuts can be precisely controlled before and after installation so that it is possible to place them accurately in the desired location suggested by the navigation system.

In general, embodiments of the improved systems provided herein are preferably particularly useful in TKA and other knee arthroplasties, but are not limited to these surgical applications. Such embodiments include at least some of the following properties: they are easy to use and manufacture, minimize tissue damage, simplify surgical procedures, are robust, can withstand multiple surgeries and required sterilization treatments, are versatile, allow for faster healing with fewer complications, require less post-surgical immobilization, are simple to use so as to require less operator training, and also less costly to produce and operate.

Disclosed herein are preferred embodiments of the systems and methods according to certain aspects of the present invention. Numerous modifications or alterations may be made without departing from the spirit and the scope of the invention.

### BRIEF DESCRIPTION OF THE DRAWINGS

FIG. 1 is a schematic three-dimensional view of an embodiment of an improved system for cutting bones during knee surgery, wherein the systems comprises a guided saw and guiding pins.

FIG. 2 is a schematic three-dimensional view of an embodiment of an improved system for cutting bones during knee surgery, wherein the systems comprises a guided saw and a guiding block.

### DETAILED DESCRIPTION

Advantages according to aspects and embodiments of the present invention are achieved by providing improved systems and methods for accurately cutting bones during orthopedic procedures. Some embodiments of the systems provided herein are adapted for preparation of a bone of a patient during TKA. More specifically, such embodiments of the system provided herein are adapted for resection of distal femur in preparation of installation of the femoral prosthetic components during TKA. However, the application principles and structures illustrated herein by the disclosed embodiments of the present invention are not limited to resection of distal femur and are not limited to TKA. Various other uses of the devices according to aspects and embodiments of the present invention are envisioned, such as, but not limited to, use in joint arthroplasty, including various knee arthroplasties, and for resection of bone tissue in any surgical procedure where precise and accurate cuts are beneficial.

An embodiment of an improved system for cutting a bone during TKA comprises: a cutting device, such as, but not limited to, a saw, wherein the cutting device comprises a cutting element adapted to resect bone. The cutting device is adapted for positioning at a patient's bone with the help of one or more guiding devices. The one more guiding devices are adapted to be connected to the bone. An embodiment of the improved system comprises link adapted to connect to guiding device to the cutting device, wherein the link is adapted to constrain movement of the cutting device; whereby the cutting device is permitted to be moved in a manner that causes the cutting element to resect the bone. In other words, the system is adapted for the guiding device to connect to or engage the cutting device and control its movement. The cutting device is, in turn, adapted for being connected to or engaged by and controlled by the guiding device.

Guiding devices according to embodiments of the systems provided herein are adapted to be stabilized at the patient. Such guiding devices can include structures for attachment to the bone, such as openings or apertures for inserting attachment pins or screws, spikes, or the like. Such guiding devices can also be fashioned as structures for attachment to the bone, such as openings or apertures for inserting attachment pins or screws, spikes, or the like. Attaching or affixing the guiding devices to the patient can be performed in a variety of ways, including percutaneous attachment, direct attachment to the bone, or by engaging a structure or a surgical device fixed relative to the patient, such as, but not limited to, an anchor post or a intramedullary rod inserted into a bone. In general, stabilization of a device with respect to the patient is not limited to attaching or affixing the device to the patient, but can be accomplished by minimizing their relative movement with respect to each other using any appropriate principle or mechanism. For example, a device and a patient can be stabilized separately with respect to the surgical table. Multiple stabilization structures can be provided to be employed at the discretion of a user.

Guiding devices, cutting devices, or both, according to certain embodiments of the present invention, are adapted for releasable engagement, connection or association with each other. In some aspects and embodiments, guiding devices comprise one or more guiding features adapted for association with cooperating structure connected to or associated with the cutting device. Guiding devices, cutting devices, or both, according to certain aspects and embodiments of the present invention, can also incorporate structures for adjustments of their position and/or orientation in at least one degree of rotational freedom and at least one degree of translational freedom with respect to the patient's bone. Some aspects and embodiments of the present invention provide multiple adjustment capabilities to the components of the improved systems without increasing their size or number of components. In certain embodiments, the adjustment capabilities include adjustments in one or more of superior/inferior, medial/lateral, or anterior/posterior translations, varus/valgus angle, flexion/extension angle, or axial rotation. Providing multiple adjustment capabilities for the same or different degrees of freedom is envisioned so that mechanisms best suited for each adjustment step can be employed. Providing mechanisms for both gross and fine adjustment control in the same system allows for more precise control of the location of the cutting block than that allowed by the conventional cutting blocks. It is also advantageous in computer-assisted surgical applications. For example, during computer-assisted surgery, the user provisionally locates the component of the system using conventional anatomical landmarks, and then fine-tunes the position using navigational feedback from the computer functionality.

In some embodiments, the structures and devices provided herein comprise several parts moveable relative to one another, thereby allowing for change of position of the parts with respect to each other and the bone. The change of position can be translational or rotational or both. The moving parts are connected by one or more structures, including but not limited to, interlocking parts, rail/slot structures, t-slots, clamps, screws, pins, racks, linear ways, gears, or ball-and-socket joints. The systems and devices of certain embodiments of the present invention also comprise structures for manipulating the relative position of the parts, such as knobs, screws, levers, or the like. The systems and devices of the disclosed embodiments of the present invention can be adapted as needed for manipulation and adjustment by a user, such as a surgeon, with or without the input of a computer functionality, an automatic, robotic, or computer assisted or aided navigating or manipulating device, or any combination or variation of the foregoing.

Improved systems according to some aspects and embodiments of the present invention are preferably adapted for performing one or more femoral cuts or resections, including, but not limited to, the cuts of the distal femur, such as, distal, axially directed anterior, axially directed posterior, anterior chamfer, or posterior chamfer cuts, or a combination thereof. In some embodiments, improved systems are adapted for performing planar cuts or resections, curved cuts or resections, or any combination or variation thereof. Integrating several such resection capabilities in the same embodiment of the system, or providing the capability to adapt the system to perform several different cuts, simultaneously or sequentially, advantageously reduces the number of components required for complete preparation of the bone. This reduction, in turn, minimizes the complexity and the size of the improved systems, rendering them particularly suitable for, although not limited to, MIS applications, CAS application, or both. Systems and devices according to some aspects and embodiments of the present invention can include aspects and components or systems for MIS or be integrated or interfaced with such systems. They can include computer functionalities, imaging or navigation functionalities, or other aspects and components or systems for CAS, or be integrated or interfaced with such systems.

### CAS Systems

In one aspect, embodiments of the present invention provide CAS systems for use by a surgeon during TKA. In one embodiment, the improved systems for accurately cutting bones are adapted for CAS. In this embodiment, the one or more guiding devices, or the saw, or both incorporate one or more surgical references for CAS navigation.

Generally, the CAS systems use various imaging and tracking devices and combine the image information with computer algorithms to track the position of the patient's anatomy, surgical instruments, prosthetic components, virtual surgical constructs such as body and limb axes, and other surgical structures and components. Some of the CAS systems use imaging systems based on CT scans and/or MRI data or on digitized points on the anatomy. Other systems align preoperative CT scans, MRIs, or other images with intraoperative patient positions. A preoperative planning system allows the surgeon to select reference points and to determine the final implant position. Intraoperatively, the CAS system calibrates the patient position to that preoperative plan, such as by using a “point cloud” technique, conventional kinematic techniques, and/or a robot to make bone preparations. Other systems use position and/or orientation tracking sensors, such as infrared sensors acting stereoscopically or otherwise, to track positions of body parts, surgery-related items such as implements, instrumentation, trial prosthetics, prosthetic components, and virtual constructs or references such as rotational axes which have been calculated and stored based on designation of bone landmarks.

As used herein, the term “position and orientation” denotes a position of an object in three-dimensional space with respect to all six degrees of freedom relative to a known coordinate system. When the object, such as a body part or a prosthetic component, is a solid member, and because the position and orientation of the fiducial marks associated with the targets are fixed, by knowing the position and orientation of the fiducials in space, the position and orientation of all surfaces on the object is also known. For example, if the position and orientation of both femoral and tibial prosthetic components is known with respect to a single reference system, the position and orientation of the components relative to one another may be determined. Prosthetic components can be navigated relative to each other in an absolute fashion, that is the computer assumes that the trials are positioned perfectly, and the gaps between the components are tracked relative to each other without the need for landmarking and without fiducials applied to the tibia and the femur. Additional landmarking, for example, for validation purposes, can be additionally be performed (for example, relative to the location of head of the femur and center of the ankle) to determine that the components were placed as desired. Similar principles can be applied to determining the position and orientation of the body parts, surgical instruments, real or virtual structures, and the like.

Processing functionality, whether standalone, networked, or otherwise, takes into account the position and orientation information as to various items in the position sensing field (which may correspond generally or specifically to all or portions or more than all of the surgical field) based on sensed position and orientation of their associated fiducials or based on stored position and/or orientation information. The processing functionality correlates this position and orientation information for each object with stored information regarding the items, such as a computerized fluoroscopic imaged file of a bone, a wire frame data file for rendering a representation of an instrumentation component, trial joint prosthesis or actual joint prosthesis, or a computer generated file relating to a rotational axis or other virtual construct or reference. The processing functionality then displays position and orientation of these objects on a screen or monitor, or heads-up display or otherwise. The surgeon may navigate tools, instrumentation, prosthetic components, actual prostheses, and other items relative to bones and other body parts to perform a surgery more accurately, efficiently, and with better alignment.

The CAS systems use the position and orientation tracking sensors to track the fiducial or reference devices associated with the body parts, surgery-related items such as implements, instrumentation, trial prosthetics, prosthetic components, and virtual constructs or references, such as limb rotational axes calculated and stored based on designation of bone landmarks. Any or all of these may be physically or virtually associated with any desired form of mark, structure, component, or other fiducial or reference device or technique that allows position or orientation, or both, of the associated item to be sensed and tracked in space, time, or both. Fiducials can be single markers or reference frames or arrays containing one or more reference elements. Reference elements can be active, such as energy emitting, or passive, such as energy reflective or absorbing, or any combination thereof. Reference elements may be optical, employ ultrasound, or employ any suitable form of electromagnetic energy, such as infrared, micro or radio waves. In general, any other suitable form of signaling may also be used, as well as combinations of various signals. To report position and orientation of the item, the active fiducials, such as microchips with appropriate field or a position/orientation sensing functionality, and a communications link, such as a spread-spectrum radio frequency link, may be used. Hybrid active/passive fiducials are also possible. The output of the reference elements may be processed separately or in concert by the processing functionality.

To locate and register an anatomical landmark, a CAS system user may employ a probe operatively associated with one or more fiducials. For example, the probe may be is triangulated in space relative to two sets of fiducials. The one or more fiducials provide information relating the landmark via a tracking/sensing functionality to the processing functionality. To indicate input of a desired point to the processing functionality, one or more devices for data input are commonly incorporated into CAS systems. The data input devices allow the user to communicate to the processing functionality to register data from the probe-associated fiducials.

A CAS system user may input data to the computer functionality by a variety of means. Some systems employ a conventional computer interface, such as a keyboard or a computer mouse, or a computer screen with a tactile interface. In some systems, the user presses a foot pedal to indicate to the computer to input probe location data. Others use a wired keypad or a wireless handheld remote. The probe may also interact with arrays, sensors, or a patient in such a way as to act like an input device.

During surgery, CAS systems employ a processing functionality, such as a computer, to register data on position and orientation of the probe to acquire information on the position and orientation of the patient's anatomical structures, such as certain anatomical landmarks, for example, a center of a femoral head. The information is used, among other things, to calculate and store reference axes of body components such as in a knee or a hip arthroplasty, for example, the axes of the femur and tibia, based on the data on the position and/or orientation of the improved probe. From these axes such systems track the position of the instrumentation, so that bone resections position the prosthetic joint components optimally, usually aligned with a mechanical axis. Furthermore, the systems provide feedback on the balancing of the joint ligaments in a range of motion and under a variety of stresses and can suggest or at least provide more accurate information than in the past about the ligaments that the surgeon should release in order to obtain correct balancing, alignment and stability of the joint, improving patient's recovery.

CAS systems can also suggest modifications to implant size, positioning, and other techniques to achieve optimal kinematics. Instrumentation, systems, and processes according to the present invention can also include databases of information regarding tasks such as ligament balancing, in order to provide suggestions to the surgeon based on performance of test results as automatically calculated by such instrumentation, systems, and processes.

CAS systems can be used in connection with computing functionality that is networked or otherwise in communication with computing functionality in other locations, whether by PSTN, information exchange infrastructures such as packet switched networks including the Internet, or as otherwise desired. Such remote imaging may occur on computers, wireless devices, videoconferencing devices or in any other mode or on any other platform which is now or may in the future be capable of rending images or parts of them produced in accordance with the present invention. Parallel communication links such as switched or unswitched telephone call connections or Internet communications may also accompany or form part of such telemedical techniques. Distant databases such as online catalogs of implant suppliers or prosthetics buyers or distributors or anatomical archives may form part of or be networked with the computing functionality to give the surgeon in real time access to additional options for implants which could be procured and used during the surgical operation.

In some aspects and embodiments, the present invention relates to a system for use by a surgeon during TKA, comprising: a tracking functionality adapted to track position and orientation of at least one fiducial attached to a knee bone such as the femur and also to devices that are adapted to cut bone, so that the position and orientation of the cutting device and the cutting element can be tracked relative to each other using computer aides surgical techniques for more accurate cutting by the surgeon. The computer may be adapted to store the data on the anatomical landmarks, the data relating to the three dimensional position and orientation of the knee prosthetic components, surgical instrumentation, body parts, and the data on the potential or existing surgical resection planes, as well as the structure and geometry of the cutting devices and cutting elements. The computer may also be adapted to calculate virtual surgical constructs, such as the surgical resection planes or the axes, based on the data stored in the memory.

### MIS Systems

In one more aspect, embodiments of the present invention provide improved bone preparation systems for TKA that are particularly useful, although not limited to, minimally invasive surgical applications. Certain aspects and embodiments of the improved systems provided herein allow the surgeon to minimize the size of the surgical incision and tissue damage, thereby reducing the surgical repairs and shortening the recovery time, and minimize damage the bone and soft tissues during installation and operation. In one embodiment, one or more components of the improved systems for accurately cutting bones are adapted for MIS by reducing their size, complexity, adapting them for use with minimally invasive surgical incisions, or any combination of the foregoing. In one embodiment, the MIS-adapted improved systems for bone preparation are adapted for CAS.

The term “minimally invasive surgery” (MIS) generally refers to the surgical techniques that minimize the size of the surgical incision and trauma to tissues. Minimally invasive surgery is generally less intrusive than conventional surgery, thereby shortening both surgical time and recovery time. Minimally invasive TKA techniques are advantageous over conventional TKA techniques by providing, for example, a smaller incision, less soft-tissue exposure, improved collateral ligament balancing, and minimal trauma to the extensor mechanism (see, for example, Bonutti, P. M., et al., Minimal Incision Total Knee Arthroplasty Using the Suspended Leg Technique, Orthopedics, September 2003). In certain aspects and embodiments, one or more components of the improved systems for cutting bone during TKA, are adapted to minimize soft-tissue exposure, trauma to the tissues, and the surgical cuts required for their use.

### System Comprising a Guided Saw and Guiding Pins

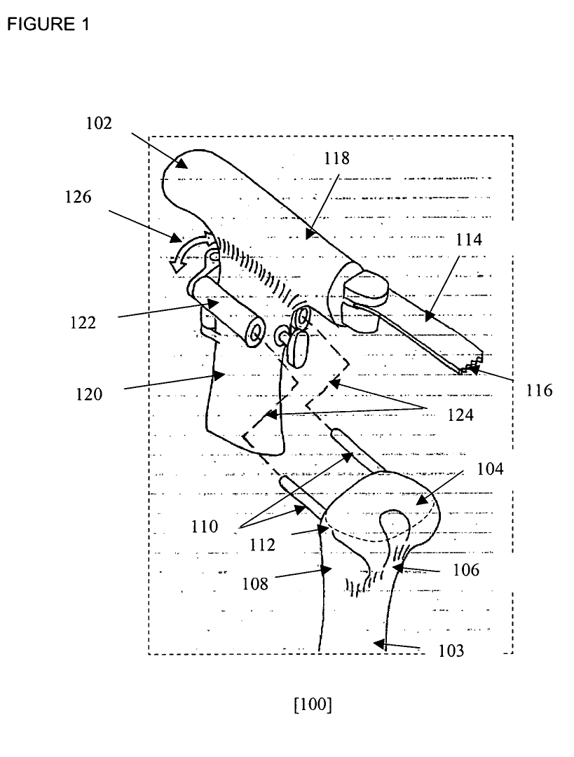

FIG. 1 schematically illustrates some aspects of an embodiment of an improved bone preparation system (100) comprising a variant of a cutting device (102), the variant also referred to as a guided saw (102). The embodiment of the system (100) is shown at a patient's distal femur (103). FIG. 1 shows distal (104), posterior (106) and medial (108) surfaces of the distal femur (103). The embodiment of the system comprises at least two guiding pins (110) that are generally or approximately parallel to each other. In the variant illustrated in FIG. 1, the guiding pins (110) are also generally or approximately parallel to the distal femoral cutting plane (112), but it is to be understood that the position and/or orientation of the pins with respect to the patient's femur (103) is variable and depends on the desirable position and/or orientation of the surgical cut, relative position and orientation of the saw (102) and the pins (110) when mutually engaged, and other factors. Generally, the pins (110) are positioned at the distal femur (103) at a desired distance from the cutting plane (112). The variant of the guided saw (102) illustrated in FIG. 1 comprises a cutting element, the cutting element also referred to as a saw blade (114) with cutting teeth (116), a housing (118), a handle (120). The cutting device (102) comprises a link (122) adapted to connect to the at least two pins (110) to the guided saw (102) wherein the link is adapted to constrain movement of the guided saw (102). In the embodiment of the system (100) illustrated in FIG. 1, the link (122) comprises at least two bushings (122) adapted to engage the pins (110).

During TKA, a user, such as a surgeon, navigates the pins (110) and installs the pins (110) by inserting them into the femur (103). For example, the pins (110) can be inserted into the femur (103) using a drilling device, such as a navigated drill guide. Drill guides are conventional and used in various surgical procedures, such as spine and trauma surgeries. After the pins are installed, the user manipulates the saw (102) to engage the pins (110) with the bushings (122). The user then manipulates the saw (102) to move the instrument towards the femur (103) following the path of the pins (110) as schematically shown by the lines (124). In other words, the pins (110) restrict the movement of and guide the saw (102). As the saw is guided by the pins, the teeth (116) of the saw blade (114) enter the bone. The user holds a saw handle (120) and cuts the bone with the saw blade (114). The saw (102) is adapted to pivot in the resection, or cutting, plane (112) with the cut as shown by the arrow (126), thereby swinging in the cutting plane (112) and covering the entire resection surface.

Reduction of the metal debris is one advantage of the embodiment of the improved system shown in FIG. 1 over the conventional systems using a cutting guide to direct the saw blade. In the conventional systems and apparatuses, moving a typically metal saw blade against the cutting guide generates metal or other material debris. The embodiment illustrated in FIG. 1, does not use a cutting block. Thus, the system does not generate a debris from the movement of the saw blade against the cutting block. This reduces tissue trauma and potential of infection.

When the embodiment of the system (100) illustrated in FIG. 1 is used in conjunction with a CAS system, or as a part of a CAS system, one or more of its components incorporate references, such as fiducials or their arrays, whose position and/or orientation is tracked by the CAS system during surgery. In a preferred embodiment of the system (100), one or more surgical references is associated with the one or more guiding pins (110). The user navigates the pins (110) with the help of a computer functionality. After navigating the pins (110) and installing them into the patient's femur (102), the user manipulates the saw (102) to engage the pins (110). Advantageously, the pins can be navigated and installed according to the user's convenience, the patient's need, and the surgical protocol at any time prior to bone preparation phase of the procedure. In the embodiment of the system (100), the user can position the saw (102) quickly and precisely at the guiding pins (110) without the help of the mechanical referencing devices and the cumbersome mounting structures used in the conventional bone-mounted knee surgery saws.

When the embodiment of the system (100) illustrated in FIG. 1 is used in a MIS procedure, the pins (110), the saw (102), or both, are advantageously adapted for installation and operation though a minimally invasive surgical incision. In one variant, the guiding pins (110) are adapted for installation and operation through a minimally invasive surgical incision by adjusting their shape and dimensions to the minimally invasive incision. After navigating the pins (110) with the CAS system and installing them into the patient's femur (103) through a minimally invasive incision, the user positions the saw (102) quickly and precisely at the guiding pins (110). Using the guiding pins (110) to position the saw (102) eliminates the need to expose the patient's tissues to accurately position of the saw blade to the anatomical references. It also eliminates the need in the mechanical referencing devices, cutting block or similar structures, all of which reduces trauma to the patient's tissues. Eliminating cumbersome mounting structures used in the conventional bone-mounted saws for knee surgery also decreases the damage to the knee.

Accordingly, the embodiment of a system (100) illustrated in FIG. 1, its variations, and methods of its use, including, but not limited to, those in conjunction with CAS or MIS, or both, systems and methods, are advantageous over the conventional bone preparations systems and methods, and provide serious benefits to the user and/or the patient.

### System Comprising a Guided Saw and a Guiding Block

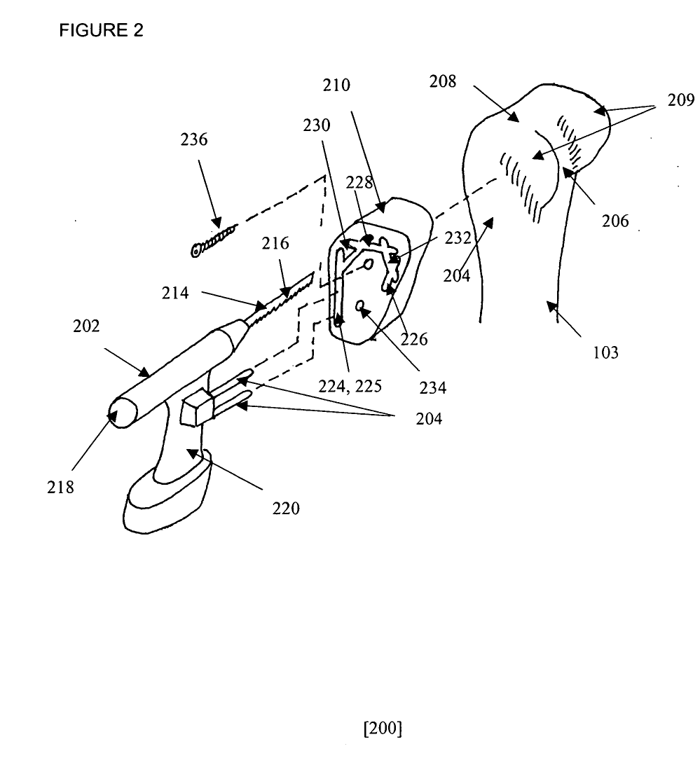

FIG. 2 schematically represents some aspects of an embodiment of an improved bone preparation system (200) comprising a variant of a cutting device also referred to as a guided saw (202). In FIG. 2, the system is shown at a patient's distal femur (203). FIG. 2 shows the medial (204), posterior (206) and distal (208) sides of the femur (203). FIG. 2 also shows the femoral condyles (209). The system comprises a guiding block (210). As shown, the guiding block (210) is being positioned at the medial side (204) of the distal femur (203), and the embodiment of the guided saw (202) is approaching the distal femur (203) at the medial side (204) Such medial approach can be particularly useful for minimally invasive knee arthroplasty. Nevertheless, the use of this embodiment need not be limited to the medial approach. For example, using the lateral or anterior approach to distal femur is also envisioned.

The embodiment of the guided saw shown in FIG. 2 (202) comprises a cutting element, wherein the cutting element is a saw blade (214) with cutting teeth (216), a housing (218), a handle (220) and at least two pins (222). As shown in FIG. 2, the pins (222) are approximately parallel to each other. The guiding block (210) comprises one or more or guiding slots (224) adapted to receive and guide the at least two pins (222) of the guided saw (202). In turn, the at least two pins (222) of the guided saw (202) are adapted to be inserted and moved within the one or more guiding slots (224).

It is to be understood that the embodiment of the system illustrated in FIG. 2 is not limited to the structures illustrated therein. More generally, the guiding block (210) comprises at least one guiding feature (224) corresponding generally to a resection to be formed on the femur (203). At least one cooperating structure (222) is connected to the cutting device (202) and adapted to track the at least one guiding feature (224) on the guiding block (210), whereby the cutting device (202) is adapted to be utilized to form at least one resection on the femoral bone corresponding to the at least one guide feature (224) on the guiding block (210). The cutting device (202) is adapted to be manipulated in a manner that allows the cooperating structure (222) to track the at least one guiding feature (224). It is to be understood that different variants of guiding features and/or cooperating structures can be used. In one non-limiting example, at least one guiding feature is one or more slots, and the at least one cooperating structure is at least one member adapted to be received in the one or more slots. In another non-limiting example, the at least one guiding feature comprises one or more structure protruding from the guiding block, and the at least one cooperating structure is adapted to receive the one or more one or more structure protruding from the guiding block.

In the embodiment shown in FIG. 2, the guiding slots (224) are adapted for guiding the guided saw (202) in anterior (225), posterior (226), distal (228), anterior chamfer (230) and posterior chamfer (232) cuts. It is to be understood that, in general, a guiding block can comprise more, fewer or different guiding slots than those shown in FIG. 2, and can guide the saw in more, fewer or different bone cuts. Variants of guiding features adapted for performing planar cuts, curved cuts, or any combinations or variations thereof, are envisioned and fall within scope of embodiments of improved systems and processes.

As illustrated in FIG. 2, the guiding block (210) comprises one or more apertures (234) for screws (236), pegs, pins or other structures for attaching the guiding block (210) to the femur (203). Accordingly, the one or more apertures (234) are adapted for receiving the screws (236), pins, pegs or other structures used for attaching the guiding block (210) to the femur (203). As shown in FIG. 2, two or more screws (236) are used. The guiding block (210) of this or similar embodiments of the improved system can be an adjustable guiding block adapted to include mechanisms for adjust the positions of the guiding slots.

During TKR, the user navigates and positions the guiding block (210) at the medial side (204) of the distal femur (203). The user attaches or secures the guiding block (210) to the femur (203) with the two or more bone screws (236). The user then inserts the pins (222) of the guided saw (202) into an appropriate guiding slot (224) of the guiding block (210). After the pins (222) are inserted into the guiding slot (224), the user moves the guided saw (202) into the femur (203) following the path of the pins (222) in the guiding slot (224). A surgeon holds the handle (220) and cuts the bone with the saw blade (214) by moving the saw (202) directed by the pins (222) in the guiding slot (224). In other words, by restricting the movement of pins (222), the guiding slot (224) restricts the movement of the saw blade (214) and guides the blade (214) in the desired resection plane. After completing the cut or as desired and/or dictated by the procedure and the patient's need, the user disengages the pins (222) from the guiding slot (224). If another cut is desired, the user inserts the pins (222) into a different guiding slot (225) and performs the cut. The uses can perform any or all of the cuts, for which the system is adapted. When any or all of the cuts are completed, the user removes the saw (202) and the block (210) from the surgical field and continues the surgical procedure.

Reduction of the metal debris is one advantage of the embodiment of the improved system shown in FIG. 2 over the conventional systems using a cutting guide to direct the saw blade. In the conventional systems and apparatuses, moving a typically metal saw blade against the cutting guide generates metal or other material debris. In the embodiment illustrated in FIG. 2, movement of the pins (222) within the guiding slots of the guiding block (210) occurs with less friction and generates less material debris than the movement of the saw blade against the cutting guide in the conventional structures.

During computer assisted surgery, the cutting block (202) is navigated into a desired position and attached to the femur with two or more screws (208), pins, or other such attachment devices.

When the embodiment of the system (200) illustrated in FIG. 2 is used in conjunction with a CAS system, or as a part of a CAS system, one or more of its components incorporate references, such as fiducials or their arrays, whose position and/or orientation is tracked by the CAS system during surgery. In a preferred embodiment of the system (200), one or more surgical references is associated with the guiding block (210). The user navigates the block (210) with the help of a computer functionality. After navigating the block (210) and stabilizing it at the patient's femur (203), the user manipulates the saw (202) to engage the pins (210). Advantageously, the block can be navigated and stabilized according to the user's convenience, the patient's need, and the surgical protocol at any time prior to bone preparation phase of the procedure. In the embodiment of the system (200), the user can position the saw (202) quickly and precisely at the guiding block (210) without the help of the mechanical referencing devices and the cumbersome mounting structures used in the conventional bone-mounted knee surgery saws.

When the embodiment of the system (200) illustrated in FIG. 2 is used in a MIS procedure, the pins (210), the saw (202), or both, are advantageously adapted for installation and operation though a minimally invasive surgical incision. In one variant, the guiding block (210) is adapted for installation and operation through a minimally invasive surgical incision by adjusting their shape and dimensions to the minimally invasive incision. In another MIS variant, the guiding block (210) can be stabilized at the patient percutaneously. In this case, the block (210) and most of the saw (202) is located outside of the patient during the procedure and there is no need to expose the tissues to accommodate most of their structures. Only the screws (236) for attaching the block and the saw blade (214) are inserted into the patient.

After navigating the block (210) with the CAS system and installing them into the patient's femur (203) through a minimally invasive incision, the user positions the saw (202) quickly and precisely at the guiding block (110). Using the guiding block (210) to position the saw (202) eliminates the need to expose the patient's tissues to accurately position of the saw blade to the anatomical references. It also eliminates the need in the mechanical referencing devices, cutting block or similar structures, all of which reduces trauma to the patient's tissues. Eliminating cumbersome mounting structures used in the conventional bone-mounted saws for knee surgery also decreases the damage to the knee.

Accordingly, the embodiment of a system (200) illustrated in FIG. 2, its variations, and methods of its use, including, but not limited to, those in conjunction with CAS or MIS, or both, systems and methods, are advantageous over the conventional bone preparations systems and methods, and provide serious benefits to the user and/or the patient.

The components of the embodiments of the improved systems described herein are manufactured according to known methods and principles. The components of the embodiments of the systems described herein may incorporate various materials, including, but not limited to, metals, ceramics, or plastics or combinations of them, various coatings, chemical elements and compounds, including organic and inorganic compounds. It is to be understood that the principles and structures of the systems comprising guided saws illustrated herein are not limited to the surgical systems, devices, and application described herein, but can be applied to a variety of systems and devices, particularly medical systems and devices.

The particular embodiments of the invention have been described for clarity, but are not limiting of the present invention. It can be readily determined that additional embodiments and features of the invention are within the scope of the appended claims and equivalents thereto. All publications cited herein are incorporated by reference in their entirety.

1. A system for resecting a femoral bone, the system comprising:
at least two pins for insertion into the femoral bone;
a cutting device having a cutting element adapted to resect bone to form a planar surface; and, a link adapted to connect to the at least two pins to the cutting device, wherein the link is adapted to constrain movement of the cutting device;
whereby the cutting device is permitted to be moved in a manner that causes the cutting element to resect the femoral bone to form a planar resection on a femoral bone without the need for a cutting guide.

2. The system of claim 1, wherein the at lest two pins are adapted for insertion into the femoral bone through one or more minimally invasive surgical incision or percutaneously.

3. The system of claim 1,
wherein the at least two pins comprise a first pin and a second pin,
wherein the first pin and the second pin are adapted for insertion into the bone approximately parallel to each other, and
wherein the link comprises a first bushing adapted to engage the first pin and a second bushing to engage the second pin.

4. The system of claim 1, wherein the planar resection on the femoral bone is one or more of an anterior femoral cut, a posterior femoral cut, a distal femoral cut, an anterior chamfer cut, or a posterior chamfer cut.

5. The system of claim 1, wherein the system further comprises:
at least two fiducials associated with the at least two pins; and,
a tracking functionality capable of tracking a position and orientation of the at least two fiducials.

6. A process for resecting a femoral bone, comprising the steps of:
inserting at least two pins into the femoral bone;
providing a cutting device having a cutting element adapted to resect the femoral bone to form a planar surface; and,
linking the at least two pins and the cutting device with a link adapted to connect to the at least two pins to the cutting device, wherein the link is adapted to constrain movement of the cutting device; and,
moving the cutting device in a manner that causes the cutting element to resect the femoral bone to form a planar resection on the femoral bone without the need for a cutting guide.

7. The process of claim 6, wherein in the step of inserting at lest two pins into the femoral bone the pins are inserted through one or more minimally invasive surgical incision or percutaneously.

8. The process of claim 6,
wherein the at least two pins comprise a first pin and a second pin, and wherein the link comprises a first bushing and a second bushing;
wherein in the step of inserting at lest two pins into the femoral bone the pins are inserted into the bone approximately parallel to each other; and,
wherein in the step of linking the at least two pins and the cutting device the first bushing engages the first pin and the second bushing engages the second pin.

9. The process of claim 6, wherein the planar surface is one or more of an anterior femoral cut surface, a posterior femoral cut surface, a distal femoral cut surface, an anterior chamfer cut surface, or a posterior chamfer cut surface.

10. The process of claim 6, further comprising the steps of:
associating at least two fiducials with the at least two pins;
providing a tracking functionality capable of tracking a position and orientation of the at least two fiducials;
providing a processing functionality; and,
using the tracking functionality and the processing functionality to navigate the at least two pins device into a desired position at the femoral bone.

11. A system for resecting a femoral bone using minimally invasive surgical techniques, the system comprising:
a guiding block adapted to be connected to a medial surface of the femoral bone, the guiding block comprising at least one guiding feature corresponding generally to at least one resection to be formed on the femoral bone;
a cutting device having a cutting element adapted to resect bone to form at least one planar surface;
at least one cooperating structure connected to the cutting device adapted to track the at least one guiding feature on the guiding block;
whereby the cutting device is adapted to be utilized to form at least one resection on the femoral bone corresponding to the at least one guide feature on the guiding block, when the cutting device is manipulated in a manner that allows the cooperating structure to track the at least one guiding feature.

12. The system of claim 11, wherein the guiding block is adapted to be connected to a medial surface of the femoral bone through one or more minimally invasive surgical incision or percutaneously.

13. The system of claim 11, wherein the at least one resection on the femoral bone is one or more of an anterior femoral cut, a posterior femoral cut, a distal femoral cut, an anterior chamfer cut, or a posterior chamfer cut.

14. The system of claim 11, wherein the at least one resection on the femoral bone is planar cut, a curved cut, or a combination thereof.

15. The system of claim 11, wherein the system further comprises:
at least one fiducial associate with the guiding block; and,
a tracking functionality capable of tracking a position and orientation of the at least one fiducial.

16. The system of claim 11, wherein the at least one guiding feature is one or more slots, and wherein the at least one cooperating structure is at least one member adapted to be received in the one or more slots.

17. The system of claim 11, wherein the at least one guiding feature comprises one or more structure protruding from the guiding block; and wherein the at least one cooperating structure is adapted to receive the one or more one or more structure protruding from the guiding block.

18. The system of claim 1 1, wherein the guiding block further comprises at least one feature for attaching the guiding block to the femoral bone.

19. The system of claim 18, wherein the at least one feature for attaching the guiding block to the femoral bone is at least one aperture adapted for receiving a screw, a pin, or a peg adapted for insertion into the femoral bone.

20. A process for resecting a femoral bone using minimally invasive surgical techniques, comprising the steps of:
providing a guiding block adapted to be connected to a medial surface of the femoral bone, the guiding block comprising at least one guiding feature corresponding generally to at least one resection to be formed on the femoral bone;
providing a cutting device having a cutting element adapted to resect bone to form at least one planar surface;
engaging the at least one guiding feature on the guiding block with at least one cooperating structure connected to the cutting device and adapted to track the at least one guiding feature;
manipulating the cutting device in a manner that allows the cooperating structure to track the at least one guiding feature, and that causes the cutting element to form the at least one resection on the femoral bone.

21. The process of claim 20, further comprising the step of connecting the guiding block to a medial surface of the femoral bone through one or more minimally invasive surgical incision or percutaneously.

22. The process of claim 20, wherein the at least one resection on the femoral bone is one or more of an anterior femoral cut, a posterior femoral cut, a distal femoral cut, an anterior chamfer cut, or a posterior chamfer cut.

23. The process of claim 20, wherein the at least one resection on the femoral bone is planar cut, a curved cut, or a combination thereof.

24. The process of claim 20, further comprising the steps of:
associating at least one fiducial with the guiding block;
providing a tracking functionality capable of tracking a position and orientation of the at least one fiducial;
providing a processing functionality; and,
using the tracking functionality and the processing functionality to navigate the guiding block into a desired position at the femoral bone.

25. The process of claim 20, wherein the at least one guiding feature is one or more slots, and wherein the at least one cooperating structure is at least one member adapted to be received in the one or more slots.

26. The process of claim 20, wherein the at least one guiding feature is one or more structure raised relative to a surface of the guiding block, and wherein the at least one cooperating structure is adapted to receive the one or more one or more structure raised relative to a surface of the guiding block.

27. The process of claim 20, wherein the guiding block further comprises at least one feature for attaching the guiding block to the femoral bone.

28. The process of claim 27,
wherein the at least one feature for attaching the guiding block to the femoral bone is at least one aperture adapted for receiving a screw, a pin, or a peg adapted for insertion into the femoral bone, and
wherein the process further comprises the step of attaching the guiding block to the femoral bone by the screw, the pin, or the peg inserted through the at least one aperture into the femoral bone.
